(19) United States
(12) Patent Application Publication
(10) Pub. No.: US 2026/0076625 A1
(43) Pub. Date: Mar. 19, 2026

(54) BIOMEDICAL SENSING SYSTEM, PATCH READING DEVICE AND BIOMEDICAL SENSING PATCH APPLICABLE THERETO

(71) Applicant: NATIONAL TSING HUA UNIVERSITY, Hsinchu (TW)

(72) Inventor: TING-WEI WANG, Hsinchu (TW)

(73) Assignee: NATIONAL TSING HUA UNIVERSITY, Hsinchu (TW)

(21) Appl. No.: 19/330,547

(22) Filed: Sep. 16, 2025

(30) Foreign Application Priority Data
Sep. 18, 2024 (TW) 113135231

(51) Int. Cl. A61B 5/00

(52) U.S. Cl. CPC A61B 5/6833; A61B 2560/0214; A61B 2562/046

(57) ABSTRACT

The biomedical sensing system includes a biomedical sensing patch and a patch reading device. The biomedical sensing patch includes a first film and an electrode. The electrode is disposed on the first film. The electrodes are configured to receive a first electromagnetic signal and generates a second electromagnetic signal in response to receiving the first electromagnetic signal. The patch reading device includes a transmission module and a reading module. The transmission module is configured to emit the first electromagnetic signal. The reading module is configured to read the second electromagnetic signal.

### TECHNICAL FIELD

The present invention relates to a biomedical sensing system, a patch reading device and a biomedical sensing patch applicable thereto; and particularly to a biomedical sensing system having a battery-free biomedical sensing patch and a patch reading device utilizing electromagnetic induction.

### BACKGROUND

With the rapid development of wireless technology and sensing technology, wearable sensors have become ubiquitous. These wearable sensors enable biomedical parameters of the human body, such as blood glucose concentration, blood pressure, heart rate, ion concentration, or activity level, to be measured through the sensors and wirelessly transmitted to a backend device (for example, a smartphone). At the backend device, the measured biomedical parameters are further analyzed or recorded in the backend device or any memory devices.

However, conventionally, most wireless sensors utilize integrated wireless transmission modules (for example, Bluetooth modules) for wireless transmission and communication. These wireless transmission modules must be powered by batteries. For the user, sensors equipped with batteries or transmission modules may be too bulky, thereby affecting mobility and comfort while using the sensors.

Therefore, there is unmet need for wearable sensors with a lighter and more convenient wearing experience. In addition, wearable sensors with simple configurations significantly reduce manufacturing costs, making them more suitable for consumable or disposable designs. Consumable or disposable designs greatly reduce safety risks associated with medical devices and are more likely to be accepted by medical regulations or general users.

### SUMMARY

Therefore, the present invention provides a biomedical sensing system to effectively overcome the problems encountered in conventional technologies.

One of the objectives of the present invention is to provide a biomedical sensing patch that does not require a battery.

One of the objectives of the present invention is to provide a suitable patch reading device.

In a specific embodiment, the present invention provides a biomedical sensing system. The biomedical sensing system includes a biomedical sensing patch and a patch reading device. The biomedical sensing patch includes a first film and an electrode. The electrode is disposed on the first film. The electrode is configured to receive a first electromagnetic signal and generates a second electromagnetic signal in response to receiving the first electromagnetic signal. The patch reading device includes a transmission module and a reading module. The transmission module is configured to emit the first electromagnetic signal. The reading module is configured to read the second electromagnetic signal.

In a specific embodiment, the present invention provides a patch reading device for the biomedical sensing system of the present invention. The patch reading device includes a transmission module and a reading module. The transmission module is configured to output the first electromagnetic signal. The reading module is configured to read the second electromagnetic signal.

In a specific embodiment, the present invention provides a biomedical sensing patch for the biomedical sensing system. The biomedical sensing patch includes a first film and an electrode. The electrode is disposed on the first film and are configured to receive the first electromagnetic signal, and in response to receiving the first electromagnetic signal, generate a second electromagnetic signal.

As described above, the battery-free biomedical sensing patch is configured, through a film-type substrate, to be attachable to a target object. The electrode on the biomedical sensing patch may sense and magnetically couple with the electromagnetic signal emitted from the patch reading device. The patch reading device may read variations in the coupled magnetic resonance of the biomedical sensing patch caused by biomedical parameters (for example, blood pressure, pulse, or ionic concentration), and thereby obtain corresponding biomedical values. The biomedical sensing patch, having a simple structure and requiring no battery, reduces discomfort for the user. Furthermore, since both the reading and excitation of the biomedical sensing patch are performed by the patch reading device, the patch reading device undertakes the overall circuit configuration, thereby significantly reducing the manufacturing cost required on the biomedical sensing patch.

### BRIEF DESCRIPTION OF THE DRAWINGS

The accompanying drawings are presented to help describe various aspects of the present invention. In order to simplify the accompanying drawings and highlight the contents to be presented in the accompanying drawings, conventional structures or elements in the accompanying drawings may be drawn in a simple schematic way or may be omitted. For example, a number of elements may be singular or plural. These accompanying drawings are provided merely to explain these aspects and not to limit them.

FIG. 1 is a schematic diagram of the biomedical sensing system of an embodiment of the present invention.

FIGS. 2A and 2B are schematic diagrams of the structure of the biomedical sensing patch of an embodiment of the present invention.

FIGS. 3A and 3B are schematic diagrams illustrating the measurement mechanism of an embodiment of the present invention.

FIG. 4 is a block diagram of the patch reading device of an embodiment of the present invention.

FIG. 5 is a block diagram illustrating the patch reading device and the backend processing unit of an embodiment of the present invention.

FIGS. 6 and 7 are schematic diagrams illustrating imaging applications of an embodiment of the present invention.

### DETAILED DESCRIPTION

Any reference to elements using terms such as “first” and “second” herein generally does not limit the number or order of these elements. Conversely, these names are used herein as a convenient way to distinguish two or more elements or element instances. Therefore, it should be understood that the terms “first” and “second” in the request item do not necessarily correspond to the same names in the written description. Furthermore, it should be understood that references to the first element and the second element do not indicate that only two elements can be used or that the first element needs to precede the second element. Open terms such as “include”, “comprise”, “have”, “contain”, and the like used herein means including but not limited to.

The term “coupled” is used herein to refer to direct or indirect electrical coupling between two structures. For example, in an example of indirect electrical coupling, one structure may be coupled with another structure through a passive element such as a resistor, a capacitor, or an inductor.

In the present invention, the term such as “exemplary” or “for example” is used to represent “giving an example, instance, or description”. Any implementation or aspect described herein as “exemplary” or “for example” is not necessarily to be construed as preferred or advantageous over other aspects of the present invention. The terms “about” and “approximately” as used herein with respect to a specified value or characteristic are intended to represent within a value (for example, 10%) of the specified value or characteristic.

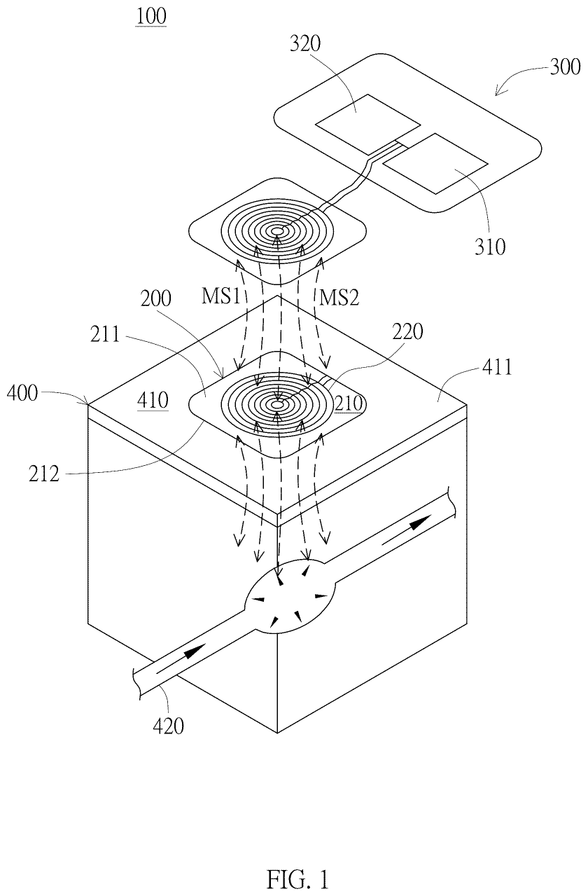

Referring to FIG. 1, FIG. 1 illustrates a biomedical sensing system 100 in accordance with one embodiment of the present invention. The biomedical sensing system 100 includes a biomedical sensing patch 200 and a patch reading device 300. The biomedical sensing patch 200 includes a first film 210 and an electrode 220. The electrode 220 is disposed on the first film 210. The electrode 220 is configured to receive a first electromagnetic signal MS1 and, in response to receiving the first electromagnetic signal MS1, to generate a second electromagnetic signal MS2. The patch reading device 300 includes a transmission module 310 and a reading module 320. The transmission module 310 is configured to emit/output the first electromagnetic signal MS1. The reading module 320 is configured to read the second electromagnetic signal MS2.

The biomedical sensing patch 200 is configured to be attachably disposed on the skin 410 of a subject 400, thereby enabling measurement of a target under test located beneath the skin 410 of the subject 400 (for example, a blood vessel 420), or a target under test located on the surface 411 of the skin 410 (for example, ions, temperature, or vibration). Specifically, the first film 210 of the biomedical sensing patch 200 may be a thin substrate formed of a flexible material with high biocompatibility, such as PLGA or PDMS. The electrode 220 may be disposed on the first film 210 by, for example, printing conductive materials. Because the first film 210 is made of a flexible material with high biocompatibility, it conforms to the surface 411 of the skin 410 of the subject 400 and reduce risks such as allergy or discomfort upon attachment. It should be noted that the electrode 220 may be disposed on either surface of the first film 210, and the present invention is not limited to the side of the first film 210 attached to the skin 410. For example, when the electrode 220 is disposed on a first surface 211 of the first film 210, the first film 210 may be attached to the skin 410 of the subject 400 through either the first surface 211 or a second surface 212 opposite to the first surface 211.

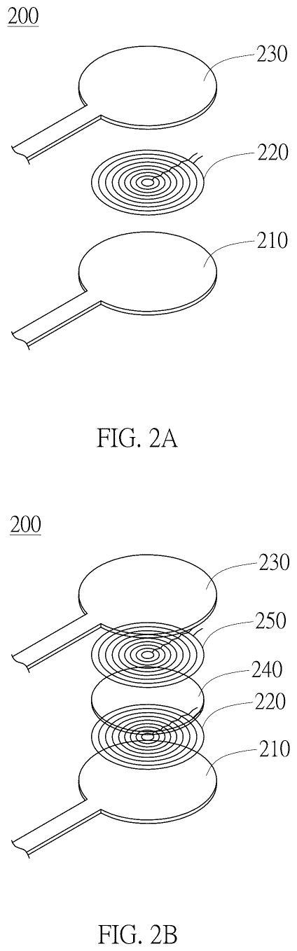

In one embodiment, referring to FIG. 2A, the biomedical sensing patch 200 further includes a second film 230 that at least covers the electrode 220. For example, when the electrode 220 is disposed on the first surface 211 of the first film 210, the second film 230 may also be disposed on the first surface 211 of the first film 210 and cover the electrode 220. Accordingly, the electrode 220 may be encapsulated by the first film 210 and the second film 230, thereby preventing issues such as deterioration or contamination of the electrode 220. The material of the second film 230 may be the same as that of the first film 210. In an embodiment, the second film 230 may be formed of an ion-exchange membrane, such that the electrode 220 is applied to ion-sensing applications. For example, ions penetrating through the second film 230 may cause a change in the impedance of the electrode 220 (e.g., a change in dielectric constant or a change in capacitance value). When the impedance of the electrode 220 changes, the second electromagnetic signal MS2 generated by the electrode 220 in response to the first electromagnetic signal MS1 also changes. Accordingly, the ion concentration sensed by the electrode 220 and the second film 230 may be obtained by the patch reading device 300. It should be noted that, in the embodiment of ion sensing, the first film 210 may also be an ion-exchange membrane, and the ion-sensing function of the present invention is not limited to the embodiment in which the second film 230 is employed.

In an embodiment, referring to FIG. 2B, the biomedical sensing patch 200 is configured to have a multilayer structure, and each layer may correspond to different electrodes 220, 250. For example, the biomedical sensing patch 200 includes an intermediate layer 240 that separates the different electrodes 220 and 250. In the embodiment, the patch reading device 300 is able to read of the electrodes 220 and 250 disposed in different layers at the same location.

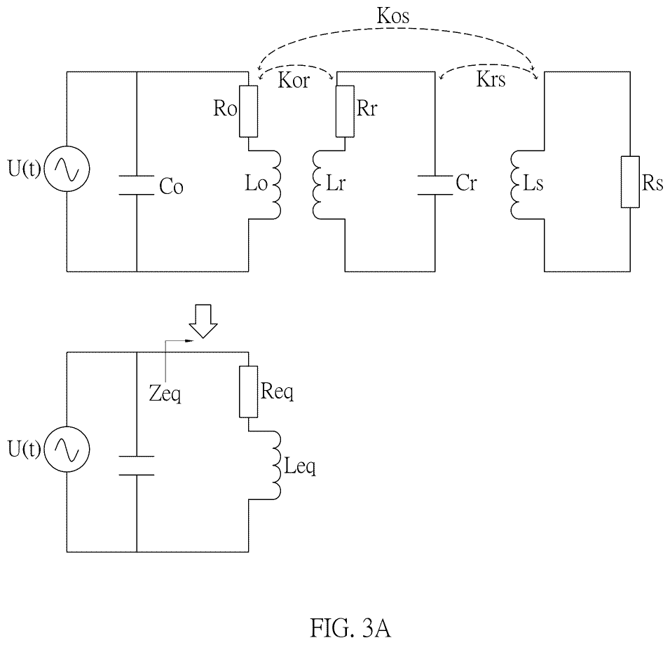

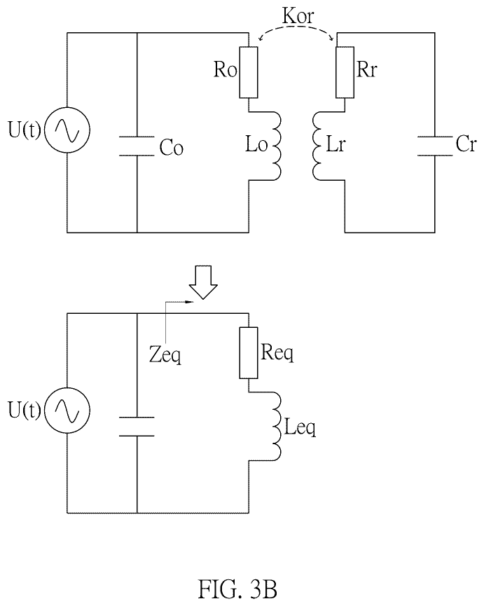

The measurement mechanism between the patch reading device 300 and the biomedical sensing patch 200 is illustrated in FIG. 3A. FIG. 3A shows a schematic circuit diagram and an equivalent circuit diagram between the biomedical sensing patch 200, the patch reading device 300, and the target under test. The patch reading device 300 emits a first electromagnetic signal MS1, and the biomedical sensing patch 200, in response to the first electromagnetic signal MS1, emits a third electromagnetic signal MS3 toward the target under test. Through the equivalent circuit diagram, it can be understood that the equivalent resistance Req, which is read by the reading circuit, is:

$$R e q = R o + \frac{{ω}^{2} Lo ( {Kos}^{2} L s Z r + K o {r}^{2} L r Z s )}{Z s Z r + {ω}^{2} K r {s}^{2} L r L s}$$

And the equivalent inductance Leq is:

$$L e q = L o - \frac{2 {ω}^{2} L o K r s K o s L s L r K o r}{Z s Z r + {ω}^{2} K r {s}^{2} L r L s}$$

Wherein, ω is the angular frequency of the excitation current source or voltage source U(t) of the patch reading device 300; Ro is the resistance of the patch reading device 300; K is a coupling coefficient, which includes Kos as the coupling coefficient between the patch reading device 300 and the target under test, Kor as the coupling coefficient between the patch reading device 300 and the biomedical sensing patch 200, and Krs as the coupling coefficient between the biomedical sensing patch 200 and the target under test; L is an inductance, which includes Lo as the inductance of the patch reading device 300, Lr as the inductance of the biomedical sensing patch 200, and Ls as the inductance of the target under test; and Z is an impedance, which includes Zr as the impedance of the biomedical sensing patch 200 and Zs as the impedance of the target under test.

Since the target under test may be a conductive substance, such as a blood vessel 420 or an ionic solution, when the state of the target under test changes (for example, changes in volume or concentration), the impedance of the target under test (inductance Ls or resistance Rs) changes accordingly. As a result, the electrode 220 on the biomedical sensing patch 200 generates a corresponding second electromagnetic signal MS2 directed toward the patch reading device 300. The reading circuit of the patch reading device 300 determines the change in the impedance of the target under test by sensing the second electromagnetic signal MS2, and thereby infer the corresponding biomedical parameter.

On the other hand, the biomedical parameter to be measured in the present invention may be directly read by the biomedical sensing patch 200. As shown in the equivalent circuit diagram of FIG. 3B, the equivalent impedance Zeq that is read by the reading circuit varies depending on the resistance Rr, inductance Lr, or capacitance Cr of the biomedical sensing patch 200. Therefore, during the measurement process, when the resistance Rr, inductance Lr, or capacitance Cr of the biomedical sensing patch 200 changes due to the biomedical parameter, the electrode 220 on the biomedical sensing patch 200 generates a corresponding second electromagnetic signal MS2 directed toward the patch reading device 300. The reading circuit of the patch reading device 300 determines the change in the impedance of the biomedical sensing patch 200 by sensing the second electromagnetic signal MS2, and thereby calculate the corresponding biomedical parameter.

It should be noted that the equivalent circuits shown in FIGS. 3A and 3B are provided merely to illustrate the sensing mechanism of the present invention and are not intended to limit the invention. For example, the reading circuit may be configured to measure changes in the impedance of the target under test as well as changes in the impedance of the biomedical sensing patch 200 itself. Therefore, the measurement scenarios described with reference to FIGS. 3A and 3B may be performed individually or in parallel.

In one embodiment of the electrode 220 layout, the electrode 220 may be arranged in a helical or concentric circular pattern to achieve improved electromagnetic coupling efficiency. In another embodiment of the electrode 220 layout, the electrode 220 may be configured as fork-shaped electrode 220. When the electrode 220 undergo a change in impedance (for example, due to vibration of the attached skin 410 or temperature variation), the change in impedance of the fork-shaped electrode 220 is detected by the reading circuit, thereby allowing calculation of the corresponding biomedical parameter. In yet another embodiment of the electrode 220 layout, a biomarker sensing layer may be disposed on the electrode 220 to capture corresponding biomarkers. For example, a biomarker sensing layer may be formed on the electrode 220 using biomarker counterparts such as glucose oxidase, antibodies, or nucleic acid primers. When the biomarker sensing layer captures the corresponding biomarker, the impedance of the electrode 220 also changes. Accordingly, the amount of the biomarker corresponding to the biomarker sensing layer (for example, the concentration) is determined. It should be noted that the biomedical sensing patch 200 may include multiple electrode 220 (for example, disposed in a multilayer structure or arranged in an array), where each electrode 220 may correspond to a different biomedical parameter and have a different configuration. In one embodiment, different configurations may be integrated into a single electrode 220 to reduce the number of electrode 220 and operational complexity. For example, an electrode 220 may be configured in a helical pattern to generate a stronger third electromagnetic signal MS3 for sensing a biomedical parameter of the target under test, and simultaneously, a biomarker sensing layer and/or an ion-exchange membrane may be disposed on the same electrode 220. The patch reading device 300 reads different biomedical parameters corresponding to a single electrode 220 by providing first electromagnetic signals MS1 of different frequencies.

Through the biomedical sensing patch 200, different biomedical parameters are measured. In particular, for electrochemical sensing applications, when ions or biomolecules react with the electrode 220, reuse may require replacement of the electrode 220 or restoration of their activity and performance through a reduction reaction. The present invention allows measurement of specific biomedical indicators through magnetic coupling or other means. When the biomedical sensing patch 200 exceeds its usage period, it can be easily replaced, or its electrode 220 are restored via electromagnetic effects or similar approaches. This significantly enhances the overall usability and safety of the system.

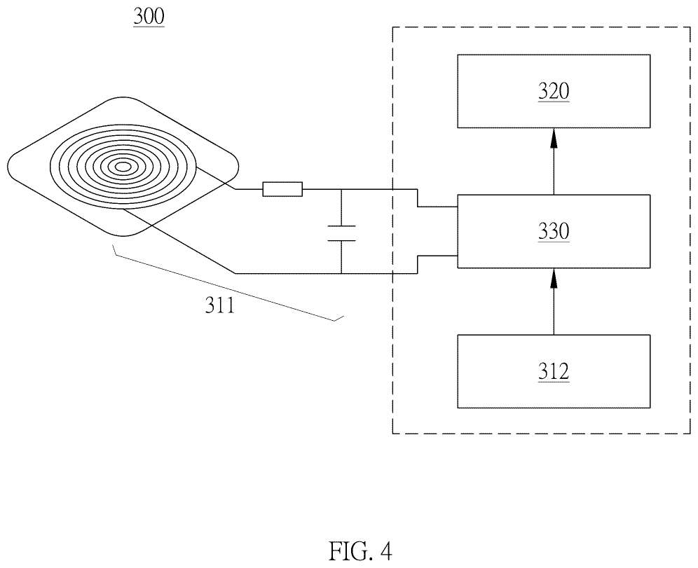

In one embodiment, referring to FIG. 4, the transmission module 310 of the patch reading device 300 may include a resonant circuit 311 and a transmission signal source 312. The transmission signal source 312 is coupled to the resonant circuit 311 and provides a transmission signal thereto. The resonant circuit 311 outputs a first electromagnetic signal MS1 in response to the transmission signal. In this embodiment, the transmission module 310 and the reading module 320 may be separated by a switching module 330 to prevent interference between the transmission of the first electromagnetic signal MS1 and the reading of the second electromagnetic signal MS2. In this embodiment, the reading module 320 may be configured to read at least one variation value caused in the resonant circuit 311 in response to receiving the second electromagnetic signal MS2. The at least one variation value may include a change in capacitance, inductance, resistance, or a combination thereof of the resonant circuit 311.

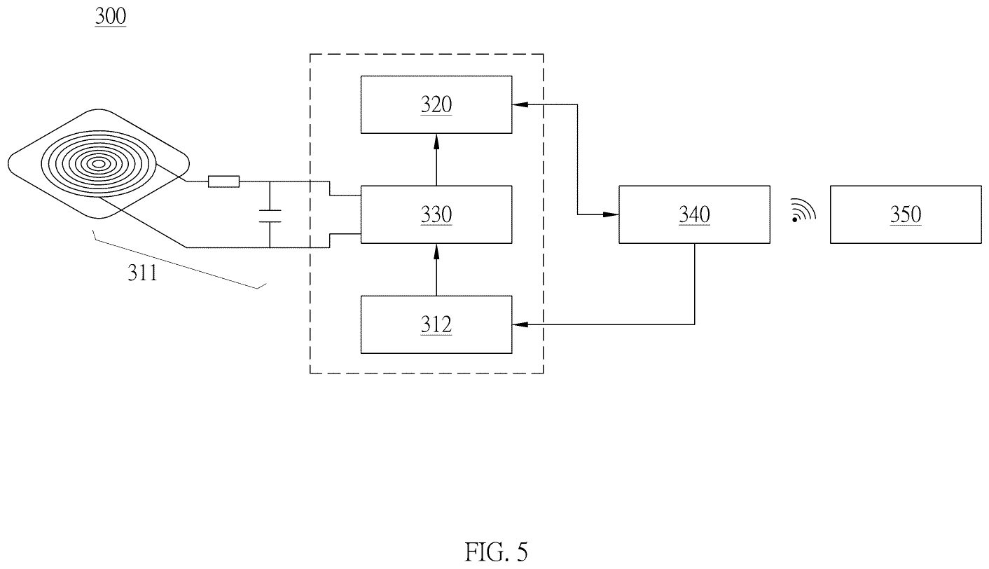

In one embodiment, referring to FIG. 5, the patch reading device 300 may further include a processor 340 and a backend processing unit 350. The processor 340 is communicatively coupled to the backend processing unit 350. The processor 340 may be configured to control the transmission module 310 and/or the switching module 330 and to receive measurement signals from the reading module 320. The processor 340 provides the measurement signals to the backend processing unit 350 via wired or wireless communication for post-processing of the measurement signals (for example, baseline removal or filtering) or for recording and storage applications.

In one embodiment of the configuration of the patch reading device 300, since the measurement between the patch reading device 300 and the biomedical sensing patch 200 is non-contact, at least a portion of the patch reading device 300 may be sealed with a sealing material. For example, the sealing material may enclose the resonant circuit 311 or the housing of the patch reading device 300. This reduces issues such as moisture ingress or internal damage caused by frequent disinfection of the patch reading device 300.

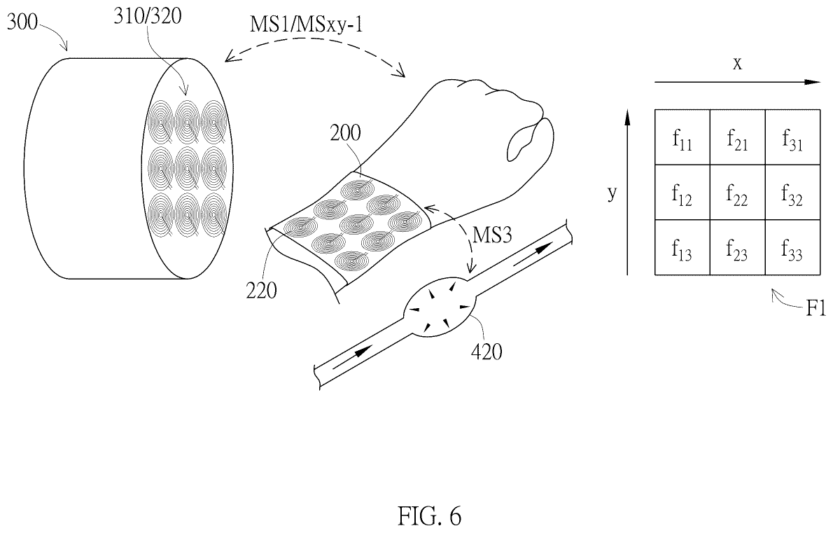

In one embodiment, the biomedical sensing system 100 may generate a sensing plane map F1 corresponding to the target under test. Specifically, referring to FIG. 6, the biomedical sensing patch 200 further includes a plurality of electrodes 220 arranged in an array. The plurality of electrodes 220 are configured, in response to receiving the first electromagnetic signal MS1, to emit a third electromagnetic signal MS3 toward the target under test to perform electromagnetic induction and generate corresponding first array position electromagnetic signals MSxy-1. For example, the plurality of electrodes 220 may have corresponding array position coordinates (x, y). When the plurality of electrodes 220 receive the first electromagnetic signal MS1 from the patch reading device 300, each of the electrodes 220 generates a corresponding third electromagnetic signal MS3 directed toward the target under test (for example, a blood vessel 420). Different positions of the target under test, after electromagnetic induction, produce different electromagnetic resonance results (i.e., the first array position electromagnetic signals MSxy-1). The reading circuit reads the electromagnetic resonance result corresponding to each of the plurality of electrodes 220. Based on the first array position electromagnetic signals MSxy-1 generated by each of the plurality of electrodes 220, the sensing plane map F1 is produced. It should be noted that the patch reading device 300 may have a number of channels equal to or greater than the number of the plurality of electrodes 220 to generate the first electromagnetic signal MS1 and receive the first array position electromagnetic signals MSxy-1. The number of channels of the patch reading device 300 may correspond to the resolution of the sensing plane map F1. In the present invention, the “number of channels” may refer, for example, to the number of first electromagnetic signals MS1 that the transmission module 310 simultaneously emits, or the number of first array position electromagnetic signals MSxy-1 that are simultaneously received. However, in one embodiment, the number of channels of the patch reading device 300 may be less than the number of the plurality of electrodes 220. In this embodiment, the sensing plane map F1 is established through scanning, translation, or similar methods.

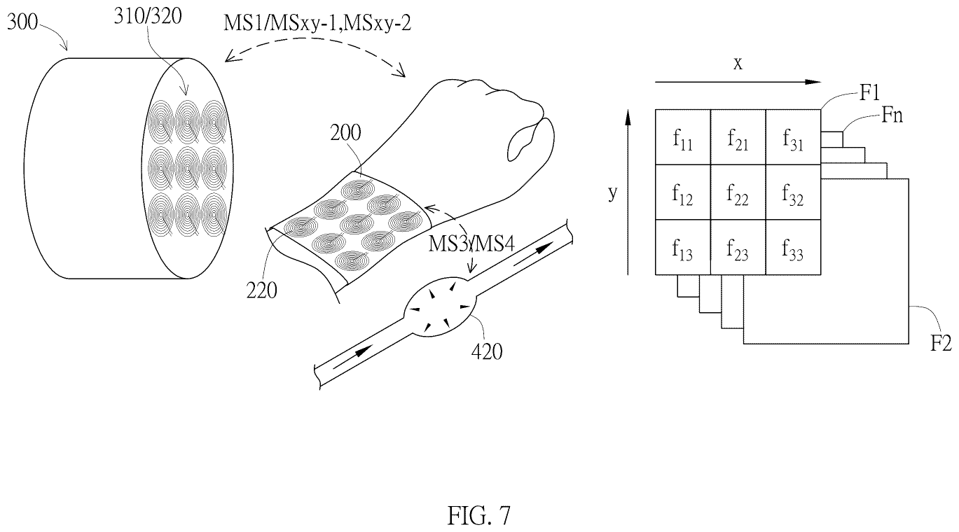

In one embodiment, as shown in FIG. 7, the biomedical sensing system 100 may further generate a sensing three-dimensional (3D) map of the target under test. Similar to generating the sensing plane map F1, the plurality of electrodes 220 are further configured, in response to receiving the first electromagnetic signal MS1, to emit a fourth electromagnetic signal MS4 having a frequency different from that of the third electromagnetic signal MS3 toward the target under test to perform electromagnetic induction, and to generate corresponding second array position electromagnetic signals MSxy-2. The reading module 320 is further configured to read both the first array position electromagnetic signals MSxy-1 and the second array position electromagnetic signals MSxy-2 to generate the sensing 3D map. For example, the patch reading device 300 may first emit the first electromagnetic signal MS1 at a first frequency, causing the plurality of electrodes 220 to generate the third electromagnetic signal MS3. Subsequently, the patch reading device 300 may emit the first electromagnetic signal MS1 at a second frequency, causing the plurality of electrodes 220 to generate the fourth electromagnetic signal MS4. Because the third electromagnetic signal MS3 and the fourth electromagnetic signal MS4 have different frequencies, they interact with the target under test at different depths. Accordingly, the second array position electromagnetic signals MSxy-2 are produced at depths different from those of the first array position electromagnetic signals MSxy-1. The reading module 320 reads the first array position electromagnetic signals MSxy-1 to generate the sensing plane map F1 and read the second array position electromagnetic signals MSxy-2 to generate the sensing plane map F2. Multiple sensing plane maps F1, F2, . . . , Fn then are combined using an overlay algorithm to generate the sensing 3D map. It should be noted that the patch reading device 300 may also adjust the electromagnetic coupling coefficient between the patch reading device 300 and the biomedical sensing patch 200 (for example, by changing the distance or the medium material) so that the plurality of electrodes 220 of the biomedical sensing patch 200 generate coupled electromagnetic signals corresponding to different depths.

In one embodiment, the plurality of electrodes 220 of the biomedical sensing patch 200 may also be configured as electrode 220 combinations having different magnetic coupling efficiencies. For example, the plurality of electrodes 220 may be arranged in a multilayer structure or disposed on the same plane (for example, in concentric circles), but are not limited thereto. By arranging multiple electrodes 220 at different layers at the same position or sharing the same center, the electrodes 220 can, in response to the first electromagnetic signal MS1 from the patch reading device 300, respectively generate coupled electromagnetic signals corresponding to different depths. In this manner, a sensing three-dimensional map is produced.

By establishing multiple measurement channels on the biomedical sensing patch 200 or the patch reading device 300, array-based measurements are realized to generate 2D or 3D images (e.g. sensing images or sensing stereograms). This allows observation of the target under test in a more intuitive and visual manner, reducing the burden on the operator to interpret numerical values.

In summary, through magnetic coupling, the patch reading device 300 and the biomedical sensing patch 200 perform inductive measurements. This enables evaluation of the biomedical parameters of the target under test beneath the skin 410 and/or the biomarkers corresponding to the biomedical sensing patch 200. Accordingly, no battery, active circuitry, chip components, or processor are required on the biomedical sensing patch 200. This allows the biomedical sensing patch 200 to be lightweight, thereby reducing the burden on the subject 400. When materials with high biocompatibility (for example, artificial skin) are used, the biomedical sensing patch 200 visually and tactilely integrates with the real skin 410 of the subject 400, providing comfortable and unobtrusive measurements. Furthermore, the simple structure of the biomedical sensing patch 200 greatly reduces manufacturing costs, making it suitable for disposable applications.

The aforementioned description of the present invention is provided to enable a person of ordinary skill in the art to make or implement the present invention. Various modifications to the present invention will be apparent to a person skilled in the art, and the general principles defined herein can be applied to other variations without departing from the spirit or scope of the present invention. Therefore, the present invention is not intended to be limited to the examples described herein, but is to be in accord with the widest scope consistent with the principles and novel features of the invention herein.

What is claimed is:

1. A biomedical sensing system, comprising:
a biomedical sensing patch including:
a first film; and
an electrode arranged on the first film, and configured to receive a first electromagnetic signal, wherein the electrode generates a second electromagnetic signal in response to receiving the first electromagnetic signal; and
a patch reading device including:
a transmission module configured to output the first electromagnetic signal; and
a reading module configured to read the second electromagnetic signal.

2. The biomedical sensing system of claim 1, wherein the transmission module includes:
a resonant circuit; and
a signal source coupled to the resonant circuit and configured to provide an excitation signal to the resonant circuit, wherein the resonant circuit outputs the first electromagnetic signal in response to receiving the excitation signal.

3. The biomedical sensing system of claim 2, wherein the reading module is configured to read at least one variation value from the resonant circuit induced by receiving the second electromagnetic signal; and wherein the at least one variation value includes the change in capacitance, inductance, resistance, or a combination thereof of the resonant circuit.

4. The biomedical sensing system of claim 1, wherein the electrode is configured to emit, in response to receiving the first electromagnetic signal, a third electromagnetic signal toward a target object.

5. The biomedical sensing system of claim 1, wherein the electrode has a sensing layer with a biomarker.

6. The biomedical sensing system of claim 1, wherein the biomedical sensing patch further includes a plurality of electrodes formed in an array; wherein the plurality of electrodes are configured to emit, in response to receiving the first electromagnetic signal, a third electromagnetic signal toward a target object to perform electromagnetic induction, and to generate corresponding first array position electromagnetic signals; and wherein the reading module is configured to generate a sensing image in response to the first array position electromagnetic signals.

7. The biomedical sensing system of claim 6, wherein the plurality of electrodes are further configured to emit, in response to receiving the first electromagnetic signal, a fourth electromagnetic signal having a frequency different from the third electromagnetic signal toward the target object to perform electromagnetic induction, and to generate corresponding second array position electromagnetic signals; and wherein the reading module is further configured to generate a sensing stereogram in response to the first array position electromagnetic signals and the second array position electromagnetic signals.

8. A patch reading device for the biomedical sensing system of claim 1, the patch reading device comprising:
the transmission module configured to output the first electromagnetic signal; and
the reading module configured to read the second electromagnetic signal.

9. The patch reading device of claim 8, wherein the transmission module includes:
a resonant circuit; and
a signal source coupled to the resonant circuit and configured to provide an excitation signal to the resonant circuit;
wherein the resonant circuit is configured to emit, in response to the excitation signal, the first electromagnetic signal.

10. The patch reading device of claim 9, wherein the reading module is configured to derive at least one variation value of the resonant circuit in response to receiving the second electromagnetic signal; and
wherein the at least one variation value is a change in capacitance, inductance, resistance, or a combination thereof.

11. The patch reading device of claim 8, wherein the reading module is configured to read first array position electromagnetic signals to form a sensing image.

12. The patch reading device of claim 11, wherein the reading module is further configured to read the first array position electromagnetic signals and second array position electromagnetic signals.

13. A biomedical sensing patch for the biomedical sensing system of claim 1, comprising:
a first film; and
an electrode arranged on the first film, and configured to receive the first electromagnetic signal, and generate, in response to receiving the first electromagnetic signal, the second electromagnetic signal.

14. The biomedical sensing patch of claim 13, wherein the electrode is configured to emit, in response to receiving the first electromagnetic signal, a third electromagnetic signal toward the target object.

15. The biomedical sensing patch of claim 13, wherein the electrode is configured to be coated with a biomarker sensing layer.

16. The biomedical sensing patch of claim 13, wherein the biomedical sensing patch further includes a plurality of electrodes formed as an array; wherein the plurality of electrodes are configured to emit, in response to receiving the first electromagnetic signal, a third electromagnetic signal toward the target object to perform electromagnetic induction on the target object, and to generate corresponding first array position electromagnetic signals.

17. The biomedical sensing patch of claim 16, wherein the plurality of electrodes are further configured to emit, in response to receiving the first electromagnetic signal, a fourth electromagnetic signal having a frequency different from the third electromagnetic signal toward the target object to perform electromagnetic induction on the target object, and to generate corresponding second array position electromagnetic signals.
